(19) United States
(12) Patent Application Publication
(10) Pub. No.: US 2007/0067017 A1
(43) Pub. Date: Mar. 22, 2007

(54) Stent with improved durability

(76) Inventor: Rainer Trapp, Graben-Neudorf (DE)

Correspondence Address: DONN K. HARMS;PATENT & TRADEMARK LAW CENTER, SUITE 100, 12702 VIA CORTINA, DEL MAR, CA, 92014, US

(21) Appl. No.: 10/571,877

(22) PCT Filed: Sep. 16, 2004
(86) PCT No.: PCT/DE04/02070

§ 371 Date: Apr. 11, 2006

(30) Foreign Application Priority Data
Sep. 16, 2003 (DE) 103 42 759.7

(51) Int. Cl. A61F 2/90

(52) U.S. Cl. 623/1.16

(57) ABSTRACT

The invention relates to a stent with improved durability. Said stent comprises a tubular grid wall extending around a longitudinal axis and consisting of elastic wall segments that are successively arranged circumferentially along the axis and are interconnected by means of connection segments. Said wall segments comprise spring elements that are interconnected by means of connection elements, forming a connection angle, and the spring elements have an undulating structure with respectively at least one crest and one trough.

The invention relates to a stent for implantation in or around a hollow organ, with several annular wall segments arranged axially in sequence, which have an elastic structure and are connected to one another by means of connecting elements.

Generic stents and their use are known in the art, and they are inserted into tubular hollow organs to expand them and keep them open. These types of stents have a grid- or spiral-shaped structure consisting of wall segments lined up axially in sequence. The wall segments themselves consist of short elastic frames connected to one another in multiple ways. Between and within the wall segments, grid openings delimited by the frames are formed which enable this structure to grow into the tissue at its implantation location. Such stents are known in the art and described, for example, in DE-A 197 46 898 or WO 03/063 733.

Stents are inserted into a hollow organ, such as blood vessels, the urinary tract, the esophagus or bile ducts, to ensure that they retain clearance and the flow of bodily fluids is not obstructed. Particularly in blood vessels, deposits frequently develop that can led to the complete occlusion of the blood vessel, which has serious health consequences. In a diseased hollow organ of this nature, this type of stent is then inserted by means of a catheter in order to support the organ wall. Self-expanding or expandable stents, for example, are frequently used; in a first state with a small diameter, they are inserted into the hollow organ and, once in this position, are allowed, in a second state with a larger diameter, to expand automatically or are actively expanded by means of an inner intrinsic elastic force. However, stents are also used when a hollow organ, such as a blood vessel, no longer has sufficient strength to retain its original shape. This leads to so-called aneurysms. In this case, there is a risk that the hollow organ, especially a blood vessel, will expand at such a diseased location and tear in the process. This leads to unwanted internal bleeding. To avoid such an internal injury, a stent, especially a coated stent, is inserted into the diseased blood vessel in such a way that the expanded segment altered by the aneurysm is bridged by the stent. In this case, the two ends of the stent seal off the healthy segment before and after the aneurysm.

In addition to adequate supporting capacity, this type of stent must also exhibit sufficient flexibility to be capable following the movements of its carrier or the blood vessels and/or hollow organs. Especially in the case of self-expanding stents, it is also necessary that the stent be compressed to the small diameter in the first state and that it then be capable of expanding to the large diameter in the second state. This flexibility is guaranteed by the fact that several elastic, annular wall segments are connected to one another by means of individual connecting elements.

However, it has been shown that when there is considerable stress, such as that which occurs during the insertion of such generic stents by means of a catheter which, during insertion, must pass through sharp turns and curves in following the respective hollow organ and/or blood vessel, the integrity and stability of such stents is not always guaranteed. Thus, it can happen that in some cases individual wall elements break, especially at their connecting points, that is, at connecting segments, elements and points, which can severely impair the function and/or hold of the stent in the body. For this reason, the goal of the invention is to provide a stent of the previously described type known in the art, which, even when severely stressed during the expansion and/or insertion of the stent into the respective hollow organ, exhibits improved durability, especially resistance to breakage. In addition, this stent should exhibit improved flexibility. This goal is achieved by the features defined in the claims.

The solution according to the invention is characterized in that, in a stent of the type known in the art, having annular elastic wall segments that contains elastic elements, these individual elastic elements are wave-shaped.

The stent according to the invention is, in its basic structure, a grid-shaped tube and/or hollow cylinder which progresses around and/or along a central axis. The stent itself is flexible and bendable along this axis, so that it can overcome turns, curves or bifurcations during insertion into a body organ. The tubular grid structure is formed by a wall provided with openings, wherein the wall contains elastic, annular wall segments progressing around the axis which are connected by means of individual connecting elements. The annular wall segments themselves consist of wall elements combined to form a ring surrounding the axis. The annular wall segments are lined up along the central axis, the individual segments being connected to one another by connecting elements. Individual annular wall segments contain elastic elements, which provide the wall segment with the desired elasticity to permit radial expansion of the stent toward the central, bendable axis, wherein the diameter of the inner tube lumen from by the wall and/or of the hollow cylinder is enlarged. In this manner, the wall segments serve the desired elastic supporting function for the hollow organ. In this regard, the wall segments preferably consist entirely of the adjacent elastic elements or frames disposed in the wall plane, which form a V-shaped or zigzag ring around the stent axis. The wall segments preferably consist of the elastic element.

The elastic elements are normally bendable frames having V-shaped terminal connections to one another via connecting points or connecting elements. The invention is characterized by a special embodiment of these bendable frames or elastic elements.

According to the invention, these elastic frames are not straight or linear, but rather wave-shaped, which results in the forces acting on these connecting points during expansion and during bending along the tube or stent axis being substantially reduced. In this regard, the wave-shaped structure advantageously progresses through the wall surface. In its simplest form, this structure consists of a single wave, that is, a wave valley and a wave peak. In this regard, the curve-shaped wave progression has at least one turning point. Naturally, it is possible, according to the invention, to form the elastic elements from several wave-shaped curves progressing in sequence. According to the invention, it is also possible to allow the wave-shaped structure to progress in a zigzag structure instead of a round, sinusoidal shape.

In another embodiment according to the invention, the elastic elements are formed in such a way that the angle formed between them at the V-shaped connecting segments is concave relative to the interior of the angle, i.e., that the angle formed tangentially by two adjacent elastic elements decreases and becomes smaller in the direction of the end of the elastic frame and/or elastic connecting element. This means that, relative to the connecting point of the elastic elements, a symmetric structure is formed that corresponds to that of the Greek letter v, when written symmetrically.

In a further development of the invention, the individual wall segments are connected to one another by means of a continuous longitudinal frame. In a special embodiment, the longitudinal frame is designed to be continuously linear, allowing it to absorb compressive strain or tensile stress in a longitudinal direction without causing a longitudinal change in the stent. In the same manner a contraction, that is, a compression of the individual wall segments, also does not produce a change in the length of the stent, because the tensile stress or compressive strain possibly occurring in this process are transferred to continuous longitudinal frame and absorbed by it.

A further embodiment of the invention is characterized by the fact that the annular wall segments comprise wall elements and/or are constructed from a plurality of such elements. These wall elements are preferably elastic elements that are alternately arranged in an angle relative to one another formed by first elastic elements and second elastic elements. This produces a zigzag elastic structure for the wall segments, so that an effective, elastic effect is achieved, which also allows for expansion in a radial direction relative to the stent axis and causes the supporting effect on the hollow organ.

The connecting elements advantageously connect either only first or second elastic elements to one another. The connection elements themselves are not elastic relative to compressive and tensile forces acting along the stent longitudinal axis and are essentially rigid, i.e., said elements, together with the elastic elements they connect, absorb these tensile and compressive forces and prevent a longitudinal change in the stent. As a result, the connected first or second elastic elements, together with the connecting segments, form the continuous longitudinal frame. To this end, the connecting segments and the elastic elements connected to them should be arranged in parallel to one another. This applies, once again, relative to a projection onto a circumferential surface of the stent. In this manner, it is ensured that the applied force acts only along the longitudinal frame and has no lateral component that could lead to an unwanted shortening or lengthening of the stent. This precludes a non-linear course allowing for longitudinal expansion or contraction, as can occur, for example, as a result of a zigzag or wave-shaped course of the longitudinal frame.

An embodiment of the invention is characterized by multiple longitudinal frames that progress in parallel to one another in a projection onto an external circumferential surface, which are disposed to be spaced relative to one another in a circumferential direction. These can consist, for example, or three or four longitudinal frames. In this manner, the wall segments are positioned relative to one another in an especially effective manner, at the same time reliably avoiding a longitudinal change in the stent.

It is also possible for the longitudinal frame to have a helical shape. This can be the case, for example, when the longitudinal frame consists of the connecting segments and the first and/or second elastic elements. This results in an especially simple structure. Nevertheless, longitudinal changes as a result of compression strain or tensile stress or compression of the stent are reliably avoided.

The connecting elements can also be thicker and/or wider than the elastic elements. Complexity is reduced, in particular, when a self-expanding stent is cut, by means of a laser, from a tubular object with a small diameter. In the first state, involving a small diameter, the connecting elements are formed, for example, to be approximately S-shaped. In the second state, involving a larger diameter, the connecting elements feature at least one component in parallel to the longitudinal frame. The connecting segments can, for example, be twice as wide as the elastic elements.

This results in an especially straightforward pattern. The stent is preferably designed to be made in one piece. This results in a stable design without unnecessary edges or predetermined breaking points.

A shape memory material, such as a so-called memory metal, namely a nickel-titanium alloy, which is also marketed under the name Nitinol, can be used as the material for the stent. Polymers of the type used in other areas of medicine for implantation into the body are also suitable for production of the stent according to the invention. Using laser beams, for example, a suitable pattern for preparing the elastic elements and the connecting elements can be cut from a tubular material with a small diameter. The expanded shape can then be stamped onto the tubular object in a manner known in the art. If the stent produced in this manner is then compressed into a state in which it has a small diameter and, by means of a catheter, for example, is introduced into a diseased blood vessel, the stent, in its position, can once again be automatically returned to the stamped shape by heating via what is known as the conversion temperature.

Other possible materials for the stent include stainless steel, plastic or so-called self-dissolving materials. These self-dissolving materials, in particular, are advantageous when a stent is not intended to be installed permanently. If self-expanding stents are not used, they can be expanded in the desired position by means of a balloon catheter, for example.

Preferably, the surface of the stent should be processed, especially refined, smoothed and/or polished. This results in a smooth surface tolerated by the body.

This inventive, wave-shaped embodiment of the elastic frames is preferably such that it does not result in a significant contribution to longitudinal elasticity, i.e., that the length of the frames does not changes along the stent longitudinal axis as a result of tensile and compressive stress.

The invention is described in greater detail on the basis of the following figures:

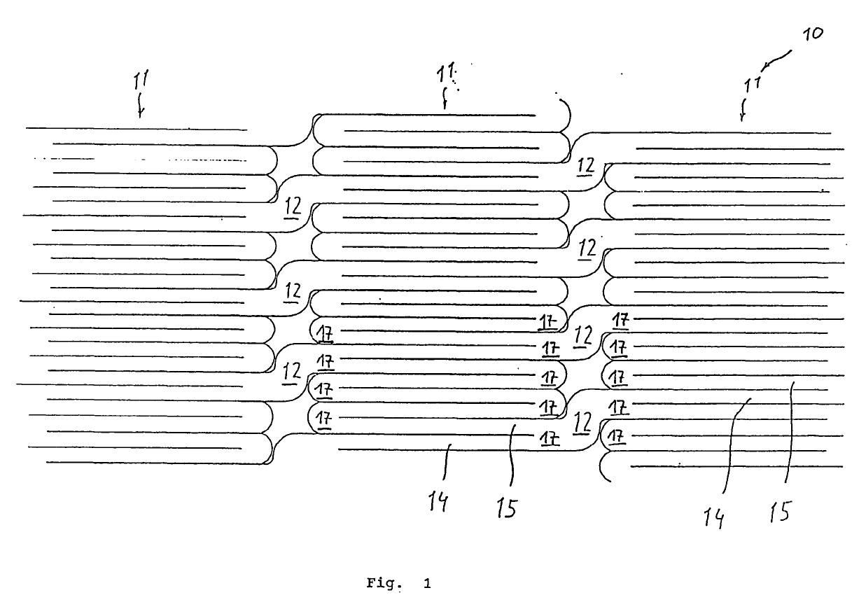

FIG. 1 shows a non-expanded, cut stent as described in the non-pre-published DE-A 102 43 136 and/or PCT/DE03/ . . . , which is based on it.

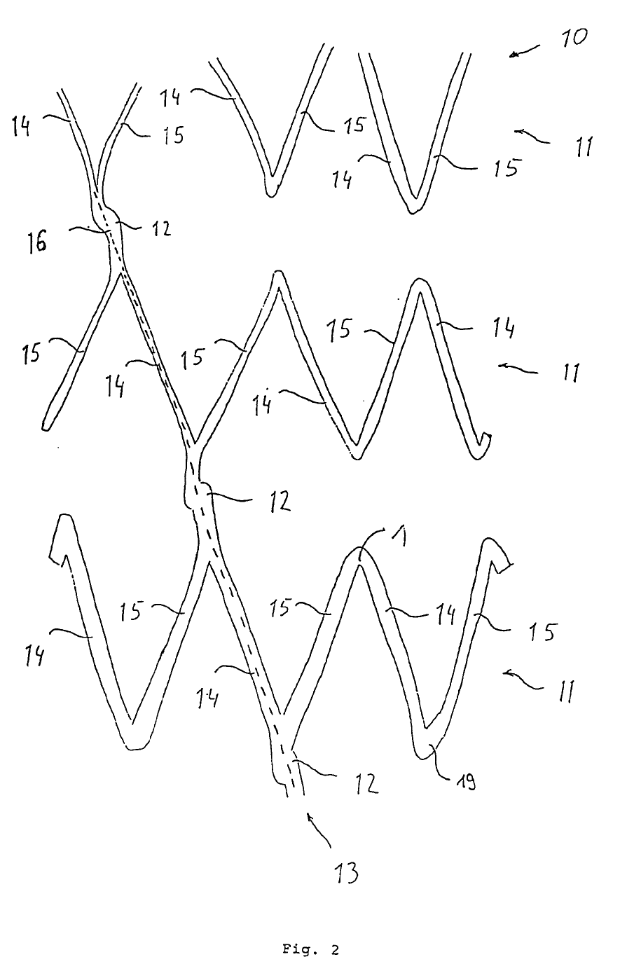

FIG. 2 shows the segment of a stent from FIG. 1 in expanded form.

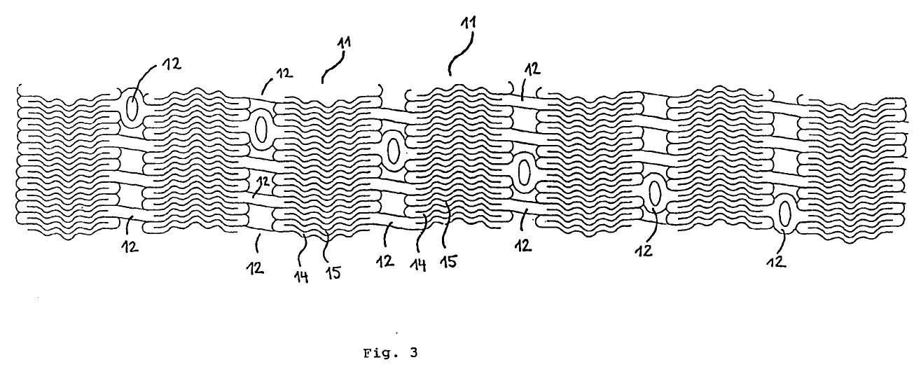

FIG. 3 shows a stent according to the invention in non-expanded form.

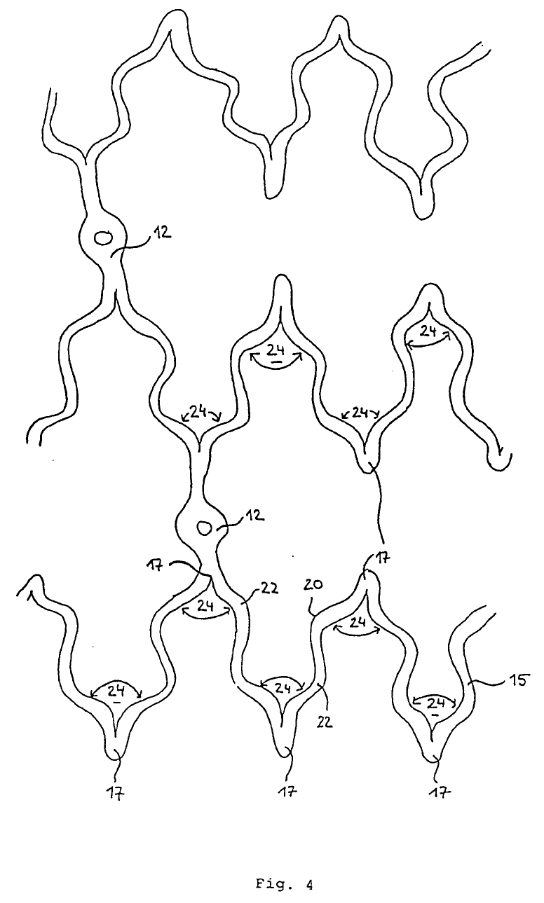

FIG. 4 shows an enlarged segment of the stent according to the invention from FIG. 3 in expanded form.

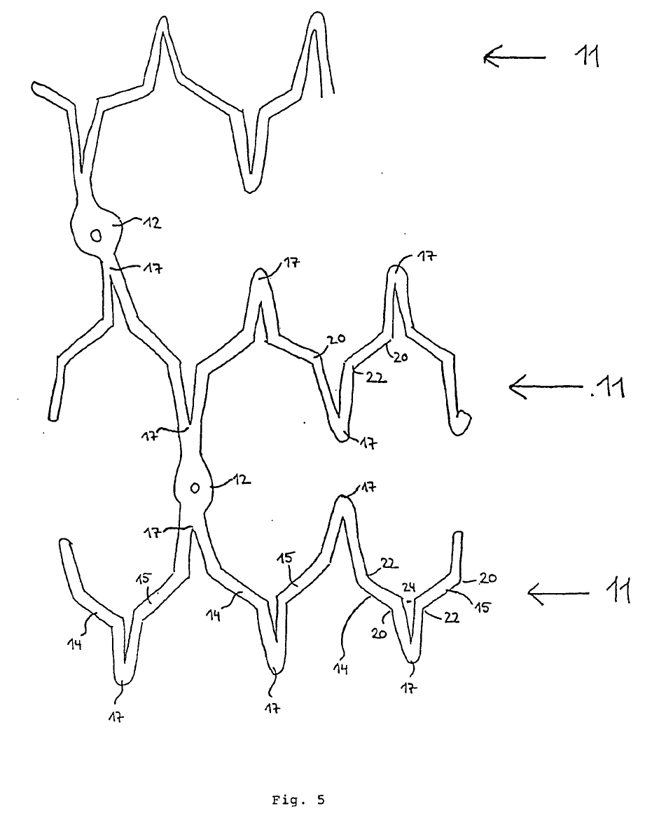

FIG. 5 shows an alternative embodiment of an expanded stent according to the invention in expanded form.

A general schematic depiction of a pattern for the production of a non-expanded stent is shown in FIG. 1. In this regard, the stent is cut, by means of laser beams, from a small tube made of a suitable material, such as a memory metal, namely a nickel-titanium alloy such as Nitinol. In this regard, FIG. 1 shows an enlarged partial view of a pattern projected onto the circumferential surface of the stent in its unwrapped form. In the interest of improved clarity, only two first elastic elements 14 and two second elastic elements 15 are provided with reference numbers in FIG. 1. As the figure indicates, in a first state following the cutting of the pattern in the small tube of memory metal, the first elastic elements 14 and the second elastic elements 15 are arranged adjacent to one another and connected to one another via connecting points and/or connecting elements 17. The first elastic elements 14 and the adjacent second elastic elements 15 are arranged to lie in parallel to one another. The figure clearly shows the roughly s-shaped form of the connecting elements 12. FIG. 1 also clearly shows that wall segments 11 arranged to be adjacent to one another are offset relative to one another by an offset corresponding to the thickness of the first elastic element 14 and the second elastic element 15. In this manner, in the first state a first elastic element 14 of a wall segment 11 is connected at its respective ends by means of connecting segments 12 with a first elastic element 14 of the adjacent wall segments 11, which is offset by the [amount of the] offset. Once the pattern is cut into the tubular blank consisting, for example, of memory metal, the stent produced in this manner is expanded in a second state having a larger diameter than the first state. This second state is then stamped onto the stent 10 in a manner known in the art. For implantation by means of a catheter, the stent 10 prepared in this manner is then compressed into a state involving a small diameter. Once it is in the desired position, the stent 10 can then be expanded once again into the stamped form by means of the so-called conversion temperature. It is also possible, however, to expand the stent 10 by means of a balloon catheter.

FIG. 2 shows an enlarged partial view of the stent 10 from FIG. 2 in expanded form. As can be deduced from the figure, each of the wall segments 11 includes first elastic elements 14 and second elastic elements 15. The first elastic elements 14 and the second elastic elements 15 are arranged at an angle relative to one another. In this manner, the first elastic elements 14 and the second elastic elements 15 form a zigzag structure. As a result, each of the wall segments 11 is formed to be elastically resilient in a radial direction.

As FIG. 2 also indicates, each of the connecting segments 12 connects adjacent first elastic elements 14 of the first wall segments 11 to one another. In this manner, the connecting segments 12 and the elastic elements 14 they connect form the longitudinal frames 13. In the figure, the individual wall segments 11 are offset relative to adjacent wall segments 11 by an offset approximately corresponding to the combined thickness of the first elastic element 14 and the second eel 15. The connecting segments 12 have an approximately s-shaped structure. This results in a direction of application of force 16, approximately parallel to the first elastic elements 14, from a first elastic element 14 to a first elastic element 14 adjacent to it. The connecting segments 12 are approximately twice as thick as the first elastic elements 14. If, in this instance, the introduction of force from a first elastic element 14 to the adjacent first elastic element 14 does not occur perfectly in parallel to their directions of extension, the lateral forces that occur can still be absorbed, to a certain extent, by this thickened version of the connecting elements 12 without resulting in a longitudinal change to the stent 10.

FIG. 3 shows a cut stent according to the invention in non-expanded form. In this regard, the elastic elements 14, 15 of the annular wall segments 11 are designed to be wave-shaped. In the example shown in the figure, the wave-shaped structure consists of three wave peaks and valleys, or of three waves connected in sequence. In principle, it is possible to provide the elastic elements with more or fewer waves. In its simplest form, the elastic element 14, 15 according to the invention consists of a wave peak and wave valley, as shown in FIG. 4, for example. In this regard, the between the connecting points 17 of the elastic elements 14, 15 features [Translator's note: there appears to be an error in the original text here, as this sentence is incomplete.] In this regard, the curved, wave-shaped course of the elastic elements 14, 15 between the connecting points 17 features at least one turning point, preferably at least three turning points. The segments 14, 15 form a connecting angle 24 at their point of connection 17 to one another. In the preferred embodiment according to the invention, the elastic elements are wave-shaped in such a way that their wave curve is concave relative to the connecting angle 24. In this regard, the size of the connecting angle gradually decreases toward connecting point 17. It has been shown that with this type of design according to the invention forces acting on the connecting points 17 and the connecting segments 12 are substantially reduced, thereby increasing the durability of the stent.

FIG. 5 shows an alternative embodiment according to the invention, in which the wave structure of the elastic elements 14, 15 is formed by a zigzag line. In this regard, the tips 20 and/or 22 correspond to the wave peaks 20, 22 of FIG. 4. In this embodiment, the angle 24 formed by the elastic elements 14, 15 at the connecting element 17 is smaller toward the connecting point 17 than in the center between the connecting points. In a preferred embodiment, the connecting elements 12 are provided with a radiopaque marking (not shown). It is especially preferred that the connecting segments 12 and the elastic elements 14, 15 form a longitudinal frame 13 which longitudinally absorbs the tensile and shear forces acting on the stent. In this regard, only connecting elements or elastic elements 14, 15 are connected in a longitudinal frame, so that several longitudinal frames progress in parallel to one another and do not intersect. In an advantageous further development, the marking circles the grid wall of the stent in a spiral shape in an axial direction, e.g., along or in parallel to the longitudinal frame 13.

### “Stent with Improved DurabilityStent with Improved Durability”

### List of Reference Numbers

10 Stent
11 Wall segment
12 Connecting element
13 Longitudinal frame
14 First elastic element
15 Second elastic element
16 Direction of application of force
17 Connecting point
20 Wave peak
22 Wave valley
24 Connecting angle

1. A stent (10) with a tubular grid wall progressing around a longitudinal axis and comprising elastic wall segments (11) arranged circumferentially in sequence along the axis and connected to one another by means of connecting segments (12), wherein the wall segments (11) comprise elastic elements (14, 15), which are connected to one another by means of connecting elements (17), while forming a connecting angle (24), characterized in that the elastic elements (14, 15) have a wave-shaped structure with at least one wave peak (20) and one wave valley (22).

2. A stent, as recited in claim 1, characterized in that the wave-shaped structure is concave, with respect to the angle (24), in the direction of the connecting element (17).

3. A stent, as recited in one of the preceding claims, characterized in that the elastic elements (14, 15) are connected to the connecting elements (12), forming a longitudinal frame (13) that progresses in an axial direction in the grid wall.

4. A stent, as recited in one of the preceding claims, characterized in that the longitudinal frame (13) encircles the wall is spiral form.

5. A stent, as recited in one of the preceding claims, characterized in that it contains several longitudinal frames (13) progressing in parallel to one another.

6. A stent, as recited in one of the preceding claims, characterized in that the stent has radiopaque markings.

7. A stent, as recited in one of the preceding claims, characterized in that the markings are arranged along a line encircling the grid wall in spiral form.

8. A stent, as recited in one of the preceding claims, characterized in that the connecting segments (12) and/or the connecting elements (17) have a greater material width than the elastic elements (14, 15).

9. A stent, as recited in one of the preceding claims, characterized in that the connections (12, 17) are at least twice as wide as elastic elements (14, 15).

10. A stent, as recited in one of the preceding claims, obtainable by cutting a hollow cylinder into a one-piece object.
